(19) United States
(12) Patent Application Publication
(10) Pub. No.: US 2004/0230166 A1
(43) Pub. Date: Nov. 18, 2004

(54) Kink resistant tube

(76) Inventors: Jason P. Hill, Brooklyn Park, MN (US); Leo M. Klisch, Maple Grove, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/788,244

(22) Filed: Feb. 26, 2004

Related U.S. Application Data
(60) Provisional application No. 60/450,555, filed on Feb. 26, 2003.

(51) Int. Cl. A61M 5/32

(52) U.S. Cl. 604/273

(57) ABSTRACT

Austenitic stainless steel tubes having good kink resistance that are prepared by heat treating cold-worked stainless steel tubes at a temperature ranging from about 550° C. to about 950° C.

### RELATED APPLICATIONS

This application claims priority from U.S. Provisional Application No. 60/450,555 filed Feb. 26, 2003, which is hereby incorporated by reference in its entirety.

### TECHNICAL FIELD

This invention relates to medical devices such as catheters, and more particularly to a stainless steel tube for use with such medical devices having good reistance to kink and fracture.

### BACKGROUND

Angioplasty is a recognized medical procedure for treating various types of endoluminal (vascular and non-vascular) disease. These procedures often utilize a flexible catheter having an inflatable balloon at the distal end and a guide wire lumen within at least a portion of the catheter. The proximal end of the catheter can consist of a relatively stiff tube so that a user can manipulate the catheter (e.g. push, pull, twist, etc.). Typically, a guide wire is inserted through the vascular system to a position near a stenosis, leaving a proximal portion of the guide wire extending from the patient. The proximal guide wire portion is threaded through the dilatation catheter guide wire lumen and the dilatation catheter is advanced through the vascular system over the guide wire to the position near the stenosis. The catheter is manipulated until the balloon is positioned across the stenosis. The balloon is then inflated by supplying fluid under pressure through an inflation lumen in the catheter to the balloon. The inflation of the balloon widens the lumen through the stenosed area by pressing the inflated balloon wall against the lesion inside the wall.

A variety of functional characteristics are generally demanded from catheters, particularly the proximal end or segment which helps manipulate and steer the catheter. These characteristics include flexibility, push strength, malleability, torquability, axial stiffness, pull strength, and kink and fracture resistance. For example, catheters have flexibility requirements that vary with the location along the catheter length. Less flexibility may be required in the proximal portion, where the catheter may lie within a relatively wide, straight vessel portion. Greater flexibility is often desired in the distal portion, where traversing relatively narrow, tortuous vessels may be needed. In the mid-region of the catheter, gradually, distally increasing flexibility is desirable, as opposed to an abrupt change from low to high flexibility.

Kinking can occur when a hypo-tube segment of a catheter is manipulated beyond its strength, causing it to bend too sharply. To avoid this, certain manufacturers provide a short strain relief that surrounds a hypo-tube segment that forms the proximate shaft of the catheter. Some designs include a strain relief or sheath formed from polymeric material that extends distally from a manifold affixed to the proximal end of a catheter shaft. Kinking is thereby inhibited where the hypo-tube exits the manifold within the strain relief. Even with such strain relief, however, catheters can still experience kinked shafts, particularly under severe manipulation during its use. The sheaths can also compromise the tensile and torsional load capacities of the device.

Current metal processing techniques can leave a tube too malleable or too flexible thereby having insufficient push strength. Other techniques can leave a tube too brittle, difficult to manipulate, and/or easily kinked. Conventional techniques can also improve kink resistance, however it can be to the detriment of malleability.

There is a need to provide a tube that can be used in medical procedures such as those that involve catheter insertions, which can be reliably manipulated without resulting in breakage and kinks. Furthermore, a metal processing technique that can provide a tube with a balanced set of performance properties is desirable.

### SUMMARY

Austenitic stainless steel tubes, which may be hypotubes, are described that have sufficient strength and flexibility to allow endoluminal manipulation (e.g., pushing, pulling, steering, torsiona forces, etc.), yet provide good resistance to fracture and kinks. For example, the tubes preferably have a center-to-center bend distance of less than 0.3 inches, measured according to the Kink Resistance Test described below. The tubes preferably have at least 70% of an austenite phase, as measured by X-ray diffraction, and are preferably prepared by heat treating cold-worked stainless steel tubes at a temperature of about 550° C. to about 950° C.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates the center-to-center bend distance results from the Kink Resistance Test, for Example 3.

FIG. 2 illustrates the Ultimate Tensile Strength test results from Example 3.

FIG. 3 illustrates the Energy to Fracture results from Example 3.

FIG. 4 illustrates the Percent Strain to Break, or Elongation, results from Example 3.

Like reference symbols in the various drawings indicate like elements.

### DETAILED DESCRIPTION

An austenitic stainless steel tube is described that is highly resistant to kinks and fractures. The tube can be used in a variety of medical devices, such as, for example, a catheter, balloon catheter, stent delivery systems (including e.g., balloon expandable and self expanding), etc. It has been found that heat treating stainless steel tubes under certain conditions can enhance the kink resistance of the material. Preferably, the outer diameter of the tube is no greater than about 0.25 inch, more preferably no more than about 0.05 inch, and the ratio of the inside diameter to the outside diameter of the tube is greater than about 0.65.

In one useful embodiment, the tube is a stainless steel tube having less than about 10% martensite phase in its microstructure, as measured by x-ray diffraction. Along with martensite, the microstructure can have austenite and ferrite phases. Preferably, the austenite phase accounts for at least 70% of the microstructure, more preferably, at least 90% of the microstructure, and even more preferably at least 98% of the microstructure. Tubes in which the martensite phase is minimized and the austenite phase is maximized can be strong, flexible, and kink resistant. In certain embodiments, the tube can have about 1% to about 5% martensite phase. In another embodiment, the amount of martensite phase present in the tube can be 2% to about 3%. Useful tubes can have a ratio of martensite to austenite of about 0.01 to about 0.25, preferably about 0.05 to about 0.15.

Magnetic properties are useful characteristics of the tube that can be measured and correlated to the performance of the tube, and are related to the microstructure of the tube. Stainless steels are generally magnetic or ferromagnetic due to the martensite and ferrite they contain. However, the material can be non-magnetic when its microstructure is fully austenite. In metastable stainless steels, such as 304 SS, deformation (e.g. cold drawing) can trigger the transformation of austenite to martensite. Ferrite phase can form during ingot solidification and welding. Furthermore, the presence of ferrite stringers in the cold drawn tube can be another cause of the higher magnetic properties.

A useful magnetic property of the tube that can be measured is magnetic susceptibility. This parameter is defined as the initial slope of a magnetization plot that charts the mass normalized magnetic moment against applied magnetic field strength. In general, the lower the magnetic susceptibility of the tube, the less prone the tube is to kinks and/or fractures. In general, tubes having relative magnetic susceptibility levels of less than about 5×10⁻³ emu/gG (electromagnetic units per gram×gauss) have useful kink- and fracture-resistant properties. Preferably, the tubes have relative magnetic susceptibility levels of about 1 to about 4×10⁻³ emu/gG The kink resistance of the tube can be measured and quantified using the Kink Resistance Test. This tests measures how closely two ends of a tube, initially bent into a U-shape, can be bent (in parallel to each other) without fracturing or kinking. Details of the test are further described in the Test Methods section, below. The distance between the center of each the tube provides a “center-to-center distance”. The tubes preferably demonstrate a center-to-center distance less than about 0.30 inches, more preferably less than about 0.20 inches.

The ultimate tensile strength (UTS) of the tube also provides a measure of its resistance to kinking and fracturing. The UTS of a material generally represents the maximum stress that a material can withstand before failure. For medical instrumentation, such as, for example, catheters, a relatively low but still workable UTS value can be desirable, as the tube could therefore have the required strength so it can be manipulated (e.g. pushed and pulled). Useful tubes for medical procedures have a UTS within the range of about 125 to about 225 ksi (kilopounds per square inch), e.g., between about 160 and 190 ksi. These ranges, however, can change, depending on the processing a tube undergoes, as well as the tube dimensions. For example, the UTS of the tube can change with wall thickness and outer diameter (OD), because the thermomechanical processing history of the material may be different from a tube with smaller dimensions. Even though the final reduction (% cold work) and heat treatment could be the same for tubes of different sizes, the upstream processing operations may add up to different overall recipes. Processing differences could manifest themselves as resultant differences in, for example, dislocation density, grain size, and phase proportions—factors that can contribute to different tensile properties. For example, a tube with 0.5 in. OD and a 0.050 in. wall thickness could have different tensile properties than a tube with 0.05 in. OD and 0.004 in. wall thickness, even though they were both subjected to the same final reduction (cold work) and heat treatment.

Another useful measure of the tube's resistance to kinks and fracture is its elongation to break. Useful tubes may have elongation to break values in the range of about 5% to about 20%, e.g., about 6% to about 20%.

The highly kink and fracture resistant tubes can be prepared by heat treating a stainless steel tube for a sufficient time to achieve softening or partial annealing. The heat treatment converts martensite to austenite. Preferably, at least 25%, and more preferably at least 50%, of the martensite present in the pre-heat treated tube is converted to austenite in the course of the heat treatment. In the process, the tube can experience stress relief, thereby improving bend fatigue life without substantially adversely affecting properties such as pushability and malleability. The longitudinal tensile yield stress can be maintained even though the longitudinal tensile strain and radial strain (e.g. crush) resistance can be increased.

The tube preferably is heat treated a temperature in the range of about 550° C. to about 950° C., more preferably about 550° C. to about 800° C., and even more preferably from about 585° C. to about 680° C. Heat treatment temperatures of about 640° C. are particularly useful. A tube placed in a heated environment for about 15 to about 30 minutes can achieve a desired level of martensite transformation to austenite. In one embodiment, the tube may be subjected to a heat soak or heat treatment for about 20 minutes. Optionally, the heat treatment can be gradually ramped to the target temperature. A cooling period can also optionally be used. The time and temperature ranges described herein can be adjusted to optimally heat treat material that may have been subjected to more or less cold work or deformation, or to account for variables such as different alloy materials and tube dimensions.

The environment in which heat treatment is performed can be any one of a variety of atmospheres conducive to partial annealing, including, but not limited to, nitrogen, argon, hydrogen, air, and combinations thereof. A vacuum chamber can also be used. Suitable pressures can range from about 10⁻⁷ torr to about 5 psig.

Any low carbon stainless steel material (e.g. including stable austenitic or metastable austenitic) can be used to prepare the heat treated tubes. The tubes can comprise amounts of martensite and austenite, therefore categorized as “austenitic.” Ferrite can also be present, although it is not necessary to achieve the performance properties of the tube. Suitable stainless steel materials that can be used in the invention include, for example, stainless steel grades 302, 304, 304L, 304 LV, 316, 316L, and 316LV. Alternative materials for articles of the invention can include other metal alloys, particularly those that are biologically compatible with a patient.

The tube may have at least one lumen extending through its entire length. The inside and outside diameters, wall thicknesses, and lengths of the tube can be within a wide range, but are preferably dimensioned for medical purposes. One category of useful tubes includes hypo-tubes, which are small diameter tubes made from stainless steel or other metallic material, such as e.g., NITINOL.

The invention will now be described by way of the following non-limiting examples:

### EXAMPLES

Test Methods

Kink Resistance Test (Test Method: 90025524)

Parameters and Equipment

| Tensile Tester: | MTS Alliance RT/5 or equivalent |
|---|---|
| Load Cell: | 22.5 lb. (100 N) |
| Test Fixture: | Kink Fixture |
| Sample Length[in]: | 7 |
| Sample End Conditions: | Free to move |
| Initial V-Block Separation [in]: | 3 |
| Kink Sensitivity [% load reduction]: | 5 |
| Displacement Speed [in/min]: | 1 |
| Data Acquisition Rate [Hz]: | 10.0 |

Seven inch samples (+/−0.5 inches) are bowed into a 3″ U-shape and placed in a fixture having upper and lower blocks. Each block has a V-groove in which the tube is set securely on. The curved end of the “U” should not extend past the end of the V-block. The ends of the sample may extend past the end of the V-block.

Run the fixture to automatically bring the blocks closer to each other, allowing them to approach slowly, while recording the amount of load (pounds) applied to the tube. The tube is compressed (e.g. bent) until a sudden drop in force, which may be indicative of a kink or fracture, is noted or when the V-blocks are too close to each other.

The gap or extension at kink is measured and converted to the center-to-center distance at kink (“K”) which is the distance between the center of each leg. Formula (I) is used to obtain the center-to-center distance:

K=G+2V−2√2r (I)

Where:

K=Shaft Center to Center Kink Distance [in]

G=Gap between the V-blocks=Start Position-Extension [in]

Start Position=3 in

Ext=Extension [in]

V=V-Block 90° Groove Depth=0.050 in

R=Shaft Outside Radius [in]

OD=Shaft Outside Diameter=2r[in]

### Example 1

Materials and Heat Treatment

Stainless steel hypotubes (304L) were heat treated by heating them in an oven maintained at one of the following temperatures: 640° C. (Example 1a) and 950° C. (Example 1b). The tubes stayed in the heated oven for 20 minutes. These tubes had a nominal inside diameter of 0.0175 in. and an outside diameter of 0.0237 in.

For comparative purposes, tubes from the same supplier were tested without a heat treatment. These tubes are identified with “NHT” which stands for non-heat treated. Samples NHT A & B were measured as having an inside diameter of 0.0178 in. and an outside diameter of 0.0237 in. Samples NHT C & D had a nominal inside diameter of 0.0200 in. and an outside diameter of 0.0264 in.

All the tubes were presumed to have been subjected to an amount of cold work/deformation, as a result of the cold drawing and formation of the tube.

Magnetic Property Testing

A 5 mm long piece of tubing was cut from a heat-treated tube using a metallography diamond sectioning saw. The test specimen was cleaned in acetone. The specimen was tested two times with a vibrating sample magnometer (VSM), according to ASTM A894. The mass of the specimen was measured with a digital gram scale. The long axis of the tubing was oriented parallel to the magnetic flux lines in the VSM. The magnetic field strength was automatically varied by the control system software and the total magnetic moment of the specimen was measured by the change in voltage in the sensing coils on the pole faces of the electromagnets.

The magnetic field was applied to the specimen starting at zero and increasing to 1.2 kG.

The magnetic field was then reversed to −1.2 kG and cycled back to 1.2 kG so that the retentivity and coercivity could be determined. Retentivity is the magnetic moment remaining in a sample when the applied field strength is returned to zero. Coercivity is the field that is applied to bring the retained moment to zero.

The magnetic moment for various applied magnetic field strengths was determined and a graph of moment values plotted against field was generated. Magnetic properties were then calculated based on the data. The slope of the magnetization graph (mass normalized magnetic moment vs. applied magnetic field strength) is considered to be relative magnetic susceptibility.

The initial slopes are relative comparisons of approximated magnetic susceptibility.

Results of the magnetic property testing are summarized in Table 1. The designation “NHT” stands for non-heat treated. It was observed that the non-heat treated hypotubes had higher relative magnetic susceptibility, magnetic saturation, and retentivity values than those that were heat treated. It is presently thought that the change in magnetic properties resulting from the heat treatment is due to the increase in an amount of austenite phase in the microstructure. The effect of the change was observed to be more pronounced with higher heat treat temperature, possibly due to increased amounts of austenite converted from retained martensite.

Tensile and Kink Resistance Testing

For tensile tests, procedures adapted from ASTM E8-96 (Test Method for Mechanical Properties: General requirements for tensile testing of Metallic Materials) and ASTM E8-96-Standard Test Methods for tension testing of Metallic Materials were used.

For kink resistance testing, the procedure described above was used (Kink Resistance Test)

Five samples from each of the tubes were tested for ultimate tensile strength and their resistance to kinking, according to the test methods set forth above. The average UTS and Kink Resistance results are provided in Table 1.

It was observed that the heat treated tubes achieved lower UTS values and center-to-center bend distance values (based on the Kink Resistance Test) than those of non-heat treated tubes. The percent elongation, or percent strain to peak load, was found to significantly increase.

TABLE 1

| | | Magnetic | | | | | | | |
|---|---|---|---|---|---|---|---|---|---|
| | | Susceptibility | Positive | | | % Strain to | | Kink | |
| | Heat | (Initial Slope | Ms | Coercivity | Retentivity | Peak Load | UTS | Load | Distance |
| Sample | Treatment | (×10⁻³ emu/gG)) | (emu/g) | (G) | (emu/g) | (elongation) | (ksi) | (lb) | (in) |
| NHT A | none | 7.6 | 24.1 | 94.9 | 4.1 | 2.74 | 186.8 | 0.323 | 0.43 |
| NHT B | none | 7.4 | 24.0 | 114.8 | 5.8 | | | | |
| NHT C | none | 9.9 | 30.5 | 70.2 | 4.9 | 2.98 | 181.1 | 0.36 | 0.55 |
| NHT D | none | 9.9 | 30.5 | 75.6 | 4.5 | | | | |
| Example | 640° C. | 1.5 | 3.3 | 149.5 | 0.5 | 7.34 | 156.2 | 0.46 | 0.17 |
| 1a | | | | | | | | | |
| Example | 640° C. | 1.6 | 3.2 | 149.1 | 0.5 | | | | |
| 1a | | | | | | | | | |
| Example | 950° C. | 0.6 | 1.5 | 93.1 | 0.1 | — | — | — | — |
| 1b | | | | | | | | | |

### Example 2

Heat Treatment

Four inch long 304L stainless steel hypotubes were heat treated in a heat treat furnace at 575° C. and 655° C. for 20 minutes in one of 3 environments: air, vacuum, or argon gas. An AVS mini-vacuum heat treat furnace was used to heat treat the hypotubes. The hypotubes were cleaned in isopropyl alcohol or acetone prior to heat treatment.

The hypotubes were heat treated in batches in the vacuum heat treat furnace. The parts were loaded into the chamber by placing them in the hearth plate holes, standing vertically. The high vacuum or inert gas partial pressure was then established, and the heat cycle was run from room temperature to the soak temperature and back to room temperature.

Heating in high vacuum was accomplished through radiated energy from the furnace heating elements to the parts. The power was cyclically applied to the heating elements to help control and maintain the desired temperature set point and reduce over- and under-shooting the setpoint temperature. In the heat treatments, the soak temperature was controlled to within +20° during the 20 minute soak period.

For the inert gas (argon) atmosphere, ultra high purity argon gas was used. Heating was achieved by conduction heating and convection currents. These methods allowed for efficient and effective transfer of heat from the heating elements to the parts. The temperature trend plots for the heat treatments in argon gas showed less cycling of temperature about the setpoint.

Intended temperature and pressure conditions for the heat treatments are provided in Table 2.

TABLE 2

| | | Heat Treatment Conditions |
|---|---|---|
| Sample | Qty In batch | (Soak Temperature, time & Atmosphere) |
| A | 6 | None (ambient) |
| B | 6 | 655° C. for 20 minutes in air |
| C | 6 | 575° C. for 20 minutes in air |
| D | 6 | 655° C. for 20 minutes under vacuum |
| E | 6 | 575° C. for 20 minutes under vacuum |
| F | 6 | 655° C. for 20 minutes in Argon |
| G | 6 | 575° C. for 20 minutes in Argon |

Tensile Tests

The MTS Sintech 1G load frame was used to tensile test the heat treated hypotubes. Tensile testing was conducted using the MTS TestWorks4 method MHT 003. The specimens had a length of 4 inch with 1 inch in each grip. An extensometer was used to determine the strain and was left on the specimen until failure. The crosshead speed was 0.1 inch/minute. The results are summarized below.

TABLE 3

| | | 0.2% Offset | Ultimate | Strain to | Strain to |
|---|---|---|---|---|---|
| | Modulus, | Yield | Tensile | Peak, % | Break % |
| SAMPLE | msi | Strength, ksi | Strength, ksi | (elongation) | (elongation) |
| A | 25.1 | 139.0 | 184.6 | 2.59 | 3.07 |
| | 27.3 | 146.3 | 197.3 | 2.33 | 2.73 |
| | 29.3 | 152.0 | 201.5 | 1.64 | 1.92 |
| Mean | 27.3 | 145.8 | 194.5 | 2.19 | 2.58 |
| Standard Deviation | 2.1 | 6.5 | 8.8 | 0.49 | 0.59 |
| C | 23.9 | 134.8 | 159.0 | 9.96 | 11.16 |
| | 25.6 | 140.3 | 165.0 | 7.50 | 7.91 |
| | 25.9 | 139.0 | 164.5 | 8.37 | 9.03 |
| Mean (575/air) | 25.1 | 138.0 | 162.8 | 8.61 | 9.36 |
| Standard Deviation | 1.1 | 2.9 | 3.3 | 1.25 | 1.65 |
| G | 33.9 | 86.1 | 130.8 | 17.73 | 18.44 |
| | 27.6 | 87.2 | 131.1 | 19.69 | 19.83 |
| | 25.9 | 93.2 | 131.6 | * | * |
| Mean (575/Ar) | 29.2 | 88.9 | 131.1 | 18.71 | 19.14 |
| Standard Deviation | 4.2 | 3.9 | 0.4 | 1.39 | 0.98 |
| E | 25.3 | 64.9 | 115.6 | 35.62 | 23.18 |
| | 29.4 | 67.2 | 119.8 | 31.34 | 32.82 |
| | 39.2 | 68.0 | 120.4 | 29.27 | 29.04 |
| Mean (575 vac) | 31.3 | 66.7 | 118.6 | 32.08 | 28.34 |
| Standard Deviation | 7.2 | 1.6 | 2.6 | 3.24 | 4.86 |
| B | 25.1 | 133.3 | 158.3 | 8.54 | 9.21 |
| | 24.7 | 134.3 | 159.3 | 8.91 | 12.21 |
| | 24.1 | 134.2 | 158.5 | 8.45 | 9.06 |
| Mean (655 air) | 24.6 | 133.9 | 158.7 | 8.63 | 10.16 |
| Standard Deviation | 0.5 | 0.5 | 0.5 | 0.24 | 1.78 |
| F | 24.3 | 60.6 | 116.0 | 34.29 | 34.99 |
| | 34.0 | 61.0 | 113.8 | 33.34 | 32.07 |
| | 22.4 | 61.0 | 114.2 | 34.48 | 35.46 |
| Mean (655 Ar) | 26.9 | 60.9 | 114.7 | 34.04 | 34.17 |
| Standard Deviation | 6.2 | 0.2 | 1.2 | 0.61 | 1.84 |
| D | 29.6 | 60.4 | 116.6 | 34.03 | 36.18 |
| | 21.6 | 59.2 | 113.3 | **18.21 | **16.74 |
| | 29.9 | 61.9 | 117.4 | 33.47 | 34.95 |
| Mean (655 vac) | 27.0 | 60.5 | 115.8 | 33.75 | 35.57 |
| Standard Deviation | 4.7 | 1.4 | 2.2 | 0.40 | 0.87 |

* On one specimen of Sample G, the extensometer slipped after the yield strength was reached, but before the ultimate strength was reached.
**On one specimen of Sample D, the strain relaxed during the test. The extensometer did not appear to have slipped, and the specimen did not appear to have slipped in the grip. The strain to peak and strain to break data was not included in the statistical calculations.

Microstructure Evaluation

For metallography, longitudinal cross-sections were prepared by casting the tubes in epoxy and grinding into the metal along the long axis of the tube to a location approximately half of the tube's outer diameter. The cross-sections were polished and etched to reveal the grain structure. Photomicrographs were taken of the typical microstructure in each tube.

The metallography results indicated that none of the heat treatments produced a fully recrystallized and equiaxed grain structure. All specimens had similar microstructures of elongated grains which are typical of as-drawn tubing products.

The metallography results indicated that all 575° C. and 655° C. heat treated tubes had similar microstructures that were typical of drawn tube products. The tensile test results indicated that the heat treatment reduced the ultimate tensile strength (UTS) and increased the percent elongation, compared to the non-heat treated specimens. These changes were more significant in the vacuum and argon treated tubes relative to those heat treated in air.

To assess (by estimation) the properties of the non-heat treated tubes, the measured UTS values were used as a reference. The non-heat treated tubes had a mean UTS that was similar to Fort Wayne Metals published properties for 304LV wire with about 60-68% cold work. The same publication lists the annealed UTS as 90 ksi and 20% cold worked UTS as 106 ksi. When the results of the tensile testing on the heat treated tubes are compared to the FWM published data for 304LV it appears that the vacuum and argon heat treatments (at 575 and 655° C.) produced mean UTS values that were similar to wire with about 20-30% cold work. The heat treatments in air produced mean UTS values that were similar to FWM properties for about 40-50% cold work. Thus, it was observed that the heat treatments reduced the strength of the cold worked wire without causing recrystallization of the cold worked grain structure; i.e., the material was not fully recrystallization annealed.

The metal material may have softened as a result of the elevated temperature exposure due to relaxation of dislocations/reduction in dislocation density and perhaps due to changes in the percentages of austenite and martensite phases present.

### Example 3

Test Protocols Used:

Tensile: ASTM E8: Tension Testing of Metallic Materials

Kink: an adaptation of EN 13868—Test Method for Kinking of Single Lumen Catheters and Medical Tubing

Materials

304 full hard stainless steel tubing (MAXXUM) was used. “Regular wall” tubes had an outside diameter of 0.025 in and an inner diameter of 0.021 in. “Thick wall” tubes had an outer diameter of 0.025 in and an inner diameter of 0.019 in. According to the supplier, the SS tubing is annealed during the draw down process when it is made to its specified size. The tubes also undergo stress relief at 426° C. (800° F.) for 20 minutes, prior to TEFLON™ coating. The TEFLON™ solvent flash off occurs at 260° C.-288° C. (500-550° F.) for 20 min (10 min ramp up, 10 min. at room temperature). A TEFLON™ cure is then performed at 260° C. for 20 minutes (10 min ramp up, 10 min. at room temperature).

“Raw” Tubing where no processing was performed except drawing them to size was also used, for comparison. These tubes had an ultimate stress (UTS) of 190 to about 200 ksi.

Heat Treatment

An oven (Hupp Co.) was used for the heat treatment of the tubes in an atmosphere of air.

The specimens were wrapped in stainless steel foil to prevent oxidation. Specimens from the tubing were prepared by cutting ten inch samples. The ends were deburred. The inner and outer diameters were measured, and the outer surface was wiped down with acetone to remove any grease or finger prints.

The oven was brought to one of the following temperatures: 260, 371, 426, 482, 510, 565, 621, 649, 732, 815, 899, 1010, and 1093° C. Note: for the 426° C. soak, the tubing was heat treated by the vendor, with no TEFLON™ heat cycle. The specimens were placed in the oven for one of the following soak times: 5, 7, 20, 30 and 60 minutes. The specimens were then removed from the oven and allowed to air cool at room temperature.

Each specimen was then treated with a simulated TEFLON™ flash off and cure by the following procedure. An oven was set to 274° C. and allowed to reach that temperature. The specimens were placed in the heated oven for 20 minutes to simulate solvent flash off. They were then removed from the oven and air cooled. The oven was allowed to come up to temperature again (274° C.) and the specimens were again placed in the oven for 20 minutes to simulate TEFLON™ cure. The specimens were then removed from the oven and allowed to air cool.

Performance Tests

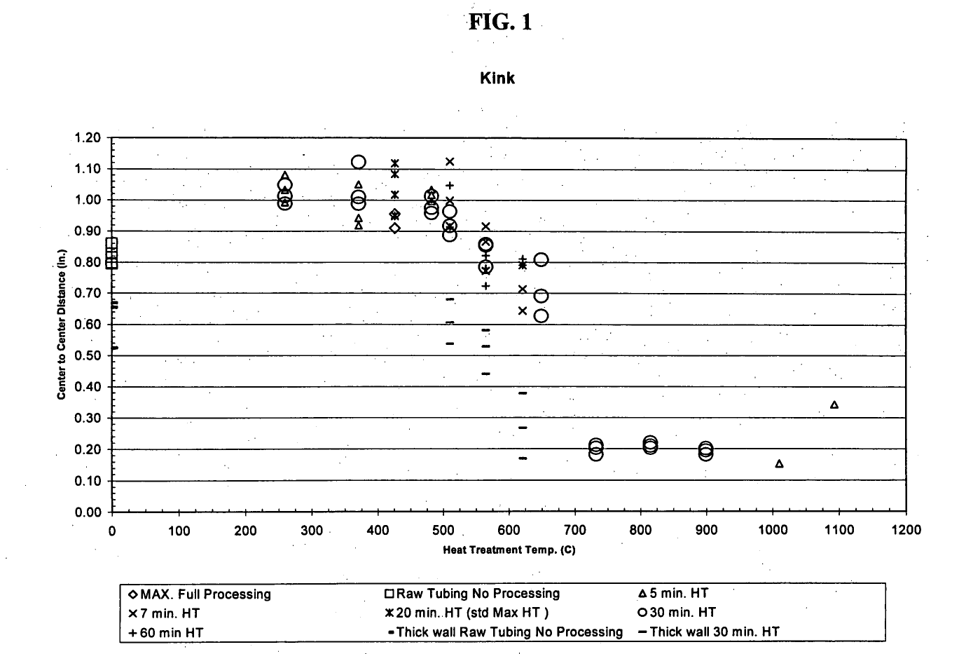

To evaluate the kink resistance of the tubes, samples were subjected to the Kink Resistance Test described above. Tensile and kink resistance test results are illustrated in FIGS. 1-4.

Referring to FIG. 1, it was observed that the center to center (c-t-c) distance increased in the 260° C. heat treated tubing, compared to that of the raw (non-heat treated) tubing. The c-t-c distance then remained steady for heat treated tubing when treated at 260° C. to 482° C. Finally, the tubes demonstrated a decrease in the c-t-c distance when heat treatment was carried out at about 510° C. to 732° C. At heat treatments beyond about 650° C., the c-t-c distance leveled off. The smaller c-t-c distance is desired for improved kink resistance performance.

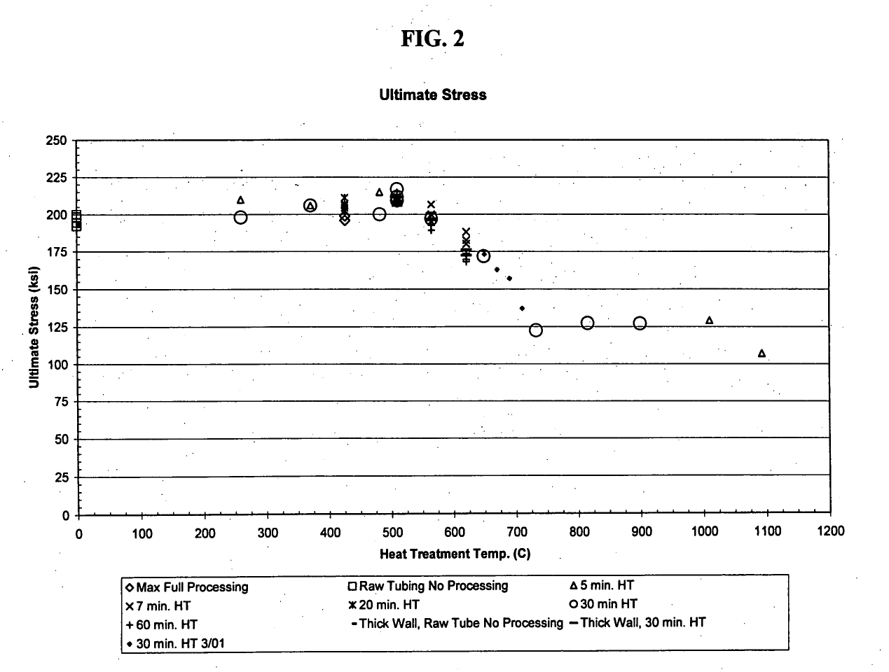

Looking at FIG. 2, the ultimate stress of the specimen heat treated at 260 to 510° C. showed an increase over that of the raw non-heat treated tubing. Heat treatments at 510 to 732° C. resulted in a decrease in ultimate stress. Values leveled off at heat treat temperatures above 732° C. A UTS between about 125 and 225 ksi demonstrated kink resistance capability.

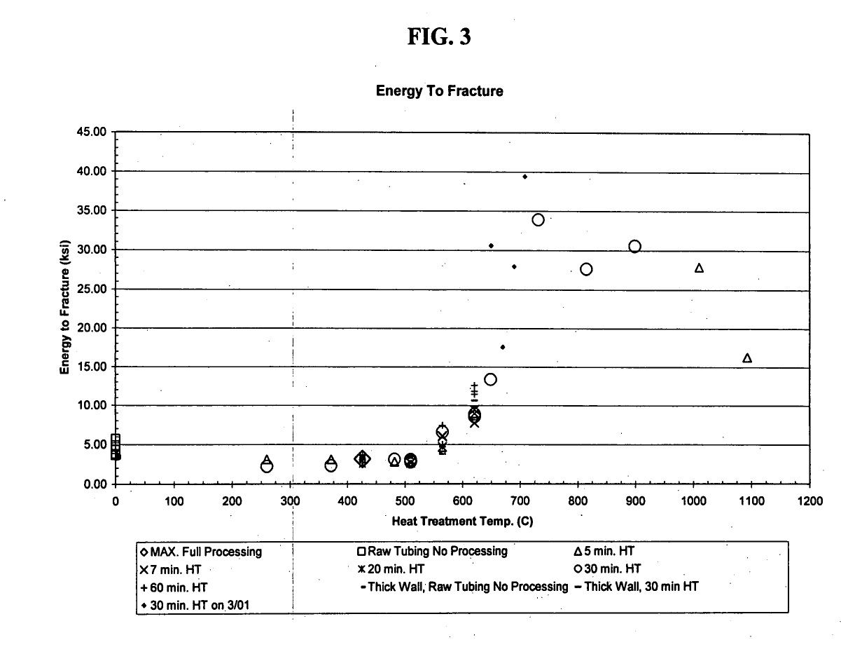

As seen in FIG. 3, the energy to fracture/percent strain at break showed a slight drop in the specimens that were heat treated at 260° C. compared to that of the raw non-heat treated tubing. These parameters then remained steady for the tubes heat treated at 260 to 510° C. At heat treat temperatures of 510° C. to 732° C., the energy and strain increased. Finally, the parameters were observed to decrease in heat treatments above 732° C.

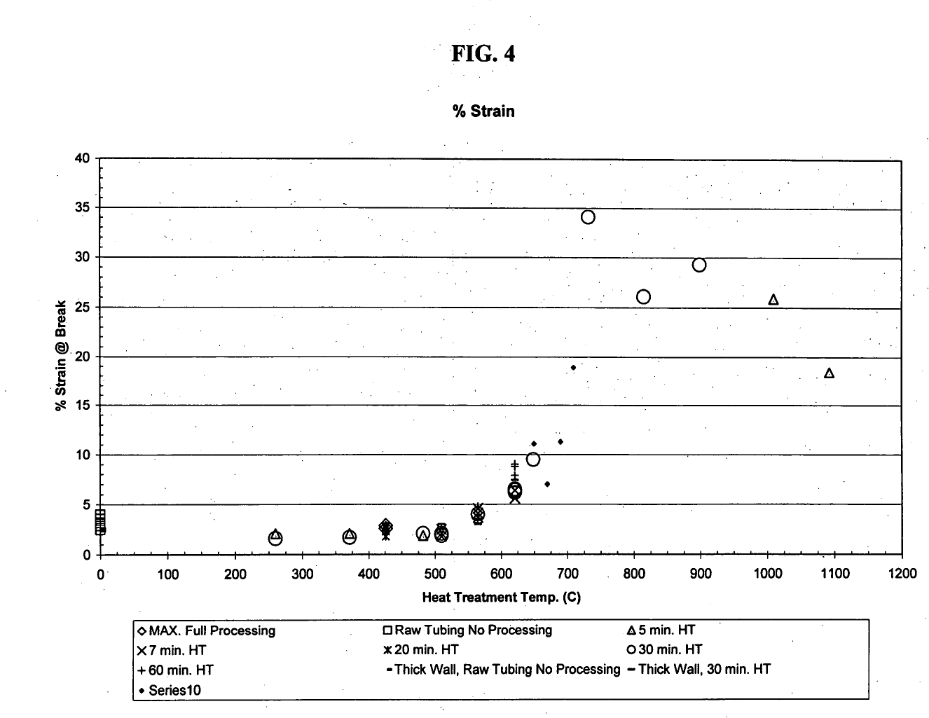

FIG. 4 illustrates how an increase in percent strain at breaking point is affected with heat treatment. At temperatures greater than about 550° C., heat treated tubes demonstrated increased levels of percent strain.

It was observed that some tubing that was heat treated above 610° C. became soft and bendable, and nearly unusable for catheter shafts. Therefore, heat treatments above about 610° C. may be undesirable for these particularly sized tubing; however, changing the dimensions and adjusting the heat treatment protocol could provide useful kink resistant product. Heat treatment at 500° C. and above showed increases in energy to break and ultimate strain, decreases in ultimate stress and improved kink resistance. Heat treatments of the work-hardened 304 tubing at 260 to 500° C. showed some increases in ultimate strain and several kinked sooner.

A number of embodiments of the invention have been described. Nevertheless, it will be understood that various modifications may be made without departing from the spirit and scope of the invention. Accordingly, other embodiments are within the scope of the following claims.

What is claimed is:

1. An article comprising an austenitic stainless steel tube having less than about 10% martensite, wherein said tube, when bent according to the Kink Resistance Test, has a center-to-center bend distance of less than 0.3 inches.

2. The article according to claim 1 wherein the tube has a center-to-center bend distance of less than about 0.2 inches.

3. The article according to claim 1 wherein the tube has an ultimate tensile strength of about 125 to about 225 kilopounds per square inch.

4. The article according to claim 1 wherein the tube exhibits a percent elongation at break of about 5% to about 20%.

5. The article according to claim 1 wherein the tube exhibits a percent elongation at break of about 6% to about 12%.

6. The article according to claim 1 wherein the tube has about 1% to about 5% martensite.

7. The article according to claim 1 wherein the tube has about 2% to about 3% martensite.

8. The article according to claim 1 wherein the tube has a ratio of martensite:austenite of about 0.01 to about 0.25.

9. The article according to claim 1 wherein the tube has at least about 70% austenite.

10. The article according to claim 1 wherein the tube has at least about 90% austenite.

11. The article according to claim 1 wherein the tube has at least about 98% austenite.

12. The article according to claim 1 wherein the tube has no more than about 5% ferrite.

13. The article according to claim 1 wherein the tube comprises stainless steel selected from the group consisting of 302, 304, 304L, 304LV, 316, 316L, and 316LV.

14. The article according to claim 1 wherein the tube has a relative magnetic susceptibility less than about 5×10⁻³ emu/gG.

15. The article according to claim 1 wherein the tube is a hypotube.

16. The article according to claim 1 wherein the tube has an outer diameter no greater than about 0.25 inches.

17. The article according to claim 1 wherein the tube has an outer diameter no greater than about 0.05 inches.

18. The article according to claim 1 wherein the tube has a ratio of the inner diameter to the outer diameter of at least about 0.65.

19. The article according to claim 1, further comprising a catheter attached to said tube.

20. An article comprising an austenitic stainless steel tube prepared by heating a cold-worked stainless steel tube at a temperature of about 550° C. to about 950° C. for a sufficient time to create a tube having at least 70% austenite.

21. The article according to claim 20 comprising heating the cold-worked stainless steel tube for a sufficient time to create a tube having at least 90% austenite.

22. The article according to claim 20 comprising heating the cold-worked stainless steel tube for a sufficient time to create a tube having at least 98% austenite.

23. The article according to claim 20 comprising heating the cold-worked stainless steel at a temperature of about 550° C. to about 800° C.

24. The article according to claim 20 comprising heating the cold-worked stainless steel at a temperature of about 585° C. to about 680° C.

25. The article according to claim 20 wherein the tube is a hypotube.

26. The article according to claim 20 wherein the tube, following heat treatment, when bent according to the Kink Resistance Test, has a center-to-center bend distance of less than 0.3 inches.

27. A method of making an austenitic stainless steel tube comprising heating a cold-worked stainless steel tube at temperature of about 550° C. to about 900° C. for a sufficient time to create an austenitic stainless steel tube having at least 70% austenite.
